(19) United States
(12) Patent Application Publication
(10) Pub. No.: US 2020/0165653 A1
(43) Pub. Date: May 28, 2020

(54) METHOD OF MEASURING FLUID SECRETION FUNCTION OF EPITHELIAL CELLS

(71) Applicants: NATIONAL UNIVERSITY CORPORATION TOKYO MEDICAL AND DENTAL UNIVERSITY, Tokyo (JP); SCREEN HOLDINGS CO., LTD., Kyoto (JP)

(72) Inventors: Mamoru WATANABE, Bunkyo-ku, Tokyo (JP); Satoru FUJII, Bunkyo-ku, Tokyo (JP); Ryuichi OKAMOTO, Bunkyo-ku, Tokyo (JP)

(21) Appl. No.: 16/604,798

(22) PCT Filed: Apr. 16, 2018
(86) PCT No.: PCT/JP2018/015761

§ 371 Date: Oct. 11, 2019

(30) Foreign Application Priority Data
Apr. 19, 2017 (JP) 2017-083118

(51) Int. Cl. C12Q 1/02; G06T 7/00; C12N 5/071; G01N 15/14

(52) U.S. Cl. CPC C12Q 1/02; G06T 7/0014; G01N 2015/1006; G01N 15/1463; G06T 2207/30024; C12N 5/0602

(57) ABSTRACT

A method of measuring a fluid secretion function of epithelial cells includes: seeding organoids established from epithelial cells in a culture receptacle and culturing the organoids in a gel in each well of the culture receptacle for a prescribed period of time; capturing a multi-pixel image of each organoid-containing well; detecting as organoids regions in the captured image that are regions surrounded by edges having at least a prescribed difference in pixel intensity value with respect to that of a background; calculating an area inside the edges for the object; selecting whether or not the object is an organoid-derived object; and determining whether or not an object selected as being derived from the organoid is subject to analysis. No cell staining product for staining the organoids is added when performing the imaging process.

### TECHNICAL FIELD

The present invention relates to a method of measuring the fluid secretion function of epithelial cells having a fluid secretion function, such as intestinal epithelial cells.

### BACKGROUND ART

Technology has been disclosed in which human intestinal epithelial cells are cultured not as individual cells, but by forming into three dimensional tissue structures called “organoids” that are pseudo crypt-villus structures configured in vivo by clumps of plural cells (“Single Lgr5 stem cells build crypt-villus structures in vitro without a mesenchymal niche” by Sato, T., et al. in Nature, 2009, 459(7244): 262-265). Namely, organoids of intestinal epithelial cells have a three dimensional shape approximating overall to a sphere or spheroidal shape of clumps with portions of voids formed such that the lumen side of the intestine is at the inside. There is also a method reported that uses such a culture system as a method to quantitatively evaluate the fluid secretion function of intestinal epithelial cells by utilizing the swelling of organoids (“A functional CFTR assay using primary cystic fibrosis intestinal organoids” by Dekkers, J. F. in Nature Med., 2013, 19(7): 939-945).

### SUMMARY OF INVENTION

### Technical Problem

In the technology described in background art above, the outer edges of organoids are marked with a fluorescent dye or the like when measuring changes in the cross-sectional area of the organoids, enabling quantitative measurements to be performed for the first time by image analysis of images obtained by imaging in such a state. This accordingly means that technology and equipment is needed to appropriately stain the subject organoids using a fluorescent dye or the like and to image the organoids. An issue therefore arises in that it is difficult to simply evaluate multiple samples in a short period of time due to needing to perform such a staining process. Moreover, stains such as fluorescent dyes or the like affect the reaction of the organoids, leading to a concern regarding the effect that this might have on the test results.

Thus an object of the present invention is to enable the fluid secretion function of epithelial cells have a fluid secretion function, such as intestinal epithelial cells, to be evaluated simply in a short period of time without needing a stain such as a fluorescent dye or the like.

### Solution to Problem

(1) First Aspect

A first aspect of the present invention is a method for measuring a fluid secretion function of epithelial cells having a fluid secretion function. The method includes a preliminary culture process, an imaging process, a detection process, a calculation process, a selection process, and a determination process. The preliminary culture process is a process of seeding organoids established from subject epithelial cells in a culture receptacle and culturing the organoids in a gel contained in each well of the culture receptacle for a prescribed period of time. The imaging process is performed after the preliminary culture process and is a process of capturing a multi-pixel image of each organoid-containing well. The detection process is a process of detecting a region as an object in the multi-pixel image captured in the imaging process, the region being surrounded by edges configured by pixels having at least a prescribed difference in pixel intensity value with respect to that of a background, the background being an average pixel intensity value of a prescribed region having a size capable of containing an organoid subject to measurement. The calculation process is a process of calculating an area inside the edges for the object detected in the detection process. The selection process is a process of selecting whether or not the object detected in the detection process is derived from an organoid. The determination process is a process of determining whether or not the object selected as being derived from the organoid in the selection process is subject to analysis. No cell staining product for staining the organoids is added when performing the imaging process.

The preliminary culture process is a process of culturing for a period of time in order to stabilize organoids in the wells of the culture receptacle, and varies according to the type of organoid employed. Examples of the culture receptacle include a multi-well plate, such as a 96 well plate for example.

The background may, for example, be determined in the following manner. A region sufficiently large with respect to the size of organoids expected to be subjected to measurement (a square of 200 μm sides, for example) is set. The average pixel intensity value is then found for all the pixels inside this region, and this is taken as the value of the background.

Pixels having a prescribed pixel intensity value or greater with respect to the background value are taken as configuring an edge of an organoid, and when a closed shape enclosed by these pixels is obtained then this shape is detected as being an object. This difference in pixel intensity value is detected as a difference in contrast between the background and the organoid. Note that this difference in pixel intensity value appears as a difference on the dark side in bright field microscopy, and appears as a difference on the light side in dark field microscopy.

The surface area of objects detected in this manner is calculated by the calculation process. The selection process is for selecting whether an object is an image derived from an organoid according to a prescribed standard or not. Moreover, the determination process is a process of determining whether or not objects selected as being organoid-derived objects are to be subject to analysis. Being subject to analysis may, for example, be determined in the determination process by determining whether “living” or not.

In the present aspect the organoids are cultured in a gel, and so the organoids are not moving due to floating, thereby enabling observations to be performed over time at the individual organoid level. Moreover, due to no cell staining product such as a fluorescent dye being added, no cell staining process is required. There is therefore not only no need to consider the effects on results of the time required for staining, but also no need to consider the effects on organoids from the cell staining product itself. Namely, observation is enabled with the organoids in a more intact state.

(2) Second Aspect

A second aspect of the present invention is the first aspect, wherein, taking a difference between a pixel intensity value of maximum brightness and a pixel intensity value of minimum brightness as 100%, in the detection process: a pixel having a difference in pixel intensity value from the background of 7.8% or greater is taken as an edge-candidate pixel; a pixel having a difference in pixel intensity value from the background of 29.2% or greater is taken as a confirmed-edge pixel; and a region surrounded by confirmed-edge pixels alone, or a region surrounded by confirmed-edge pixels and edge-candidate pixels, is detected as the object.

For example, when pixel intensity values are expressed as on an 8 bit display as is employed for ordinary image analysis, then the pixel intensity values are represented by a gray-scale with a pixel intensity value 0 representing black and a pixel intensity value 255 representing white. Under such assumptions setting a difference in pixel intensity value from the background of 7.8% or above as edge-candidate pixels is equivalent to there being a difference in pixel intensity value of 20 or greater thereto. Similarly, setting a difference in pixel intensity value from the background of 29.2% or more as the confirmed-edge pixels is equivalent a different in pixel intensity value of 75 or greater thereto. Namely, in such a case, when a closed shape is obtained enclosed by “points” that are pixels having a pixel intensity value from the background of 75 or greater, and by “lines” that are pixels between these “points” having a pixel intensity value from the background of 20 or greater, then such a closed shaped is detected as an object.

(3) Third Aspect

Furthermore, a third aspect of the present invention is the first or second aspect, wherein in the selection process: for a compactness C_{P} defined by C_{P}=P²/(4π·A), in which A (μm²) is an area of the object and P (μm) is a perimeter length of the object, an object having a compactness C_{P} of 2.0 or less is selected as being derived from the organoid.

The selection process is a process of selecting whether an object detected in the detection process is an organoid-derived object or not. The main purpose of this process is essentially to select whether or not an object is an organoid, or is an impurity other than an organoid (frankly speaking, debris). In addition to adopting the approach regarding compactness as described above, an object having a surface area occupying more than a prescribed proportion (25%, for example) of the total surface area of the well may be determined to be “debris”, and an object of the prescribed proportion or less may be selected as an object. Or, when taking a difference between a pixel intensity value of maximum brightness and a pixel intensity value of minimum brightness as 100%, then when there is a difference between the average pixel intensity value of an object to the pixel intensity value of the background of a prescribed amount or greater (25% or greater, for example), then this object may be determined to be “debris” and not selected.

(4) Fourth Aspect

A fourth aspect of the present invention is any one of the first to the third aspects, wherein in the determination process: for a circularity C_{R} defined by C_{R}=(4π·A)/E², in which A (μm²) is an area of the object and E (μm) is an edge length of the object, an object having a circularity C_{R} of 0.45 or greater is determined to be subject to analysis.

The determination process is a process that aims to determine whether or not the object is living or dead, and in addition to adopting the approach described above, an object having a surface area within a prescribed range (from 1,500 μm² to 2,400,000 μm², for example) may be determined to be living. Moreover, taking the optical density of the pixel intensity values of maximum brightness to be 0 and the optical density of the pixel intensity values of minimum brightness to be 400, then an object for which the average optical density of the pixels configuring the object is 30 or less may be determined to be a living object.

(5) Fifth Aspect

Furthermore, a fifth aspect of the present invention is any one of the first to the fourth aspects, wherein: the imaging process includes a T₀ imaging process executed immediately after the preliminary culture process, and a T_{N} imaging process executed subsequently to the T₀ imaging process after a prescribed period of time has elapsed from execution of a prescribed treatment in each of the wells; and further including an area change calculation process to calculate, for an object determined by the determination process to be subject to analysis, a change in an area of the object according to the T_{N} imaging process with respect to an area of the object according to the T₀ imaging process.

An example of the prescribed treatment is, for example, addition to the wells of a reagent subject to observation.

In the present aspect a multi-well plate may, for example, be employed as the culture receptacle, and so different types and different concentrations of reagent may be introduced into each well, enabling rapid imaging thereof for each well. Moreover, due to there being no need to consider a staining process using a cell staining product, the change in organoids can be observed for a net time (i.e. T_{N}−T₀) from a point in time (T₀) immediately prior to execution of the prescribed treatment to a point in time (T_{N}) when a prescribed period of time has elapsed from execution of the prescribed treatment.

(6) Sixth Aspect

Moreover, a sixth aspect of the present invention is any one of the first to the fourth aspects, wherein: the method employs an image analysis device that includes a stage on which the culture receptacle is placed so as to be observable under conditions appropriate for imaging the organoid, an illumination unit configured to illuminate each well from above the stage, an imaging unit configured to capture rays of light emitted by the illumination unit that have passed through each well as a multi-pixel image, an image data storage unit configured to store the multi-pixel image as image data, and a computation unit configured to perform a computation based on the image data; and the imaging process is executed by the illumination unit and the imaging unit in a state in which the culture receptacle has been placed on the stage; and the computation unit executes the detection process, the calculation process, and the determination process based on the image data stored by the image data storage unit.

(7) Seventh Aspect

A seventh aspect of the present invention is the fifth aspect, wherein: the method employs an image analysis device that includes a stage on which the culture receptacle is placed so as to be observable under conditions appropriate for imaging the organoid, an illumination unit configured to illuminate each of the wells from above the stage, an imaging unit configured to capture rays of light emitted by the illumination unit that have passed through each of the wells as a multi-pixel image, an image data storage unit configured to store the multi-pixel image as image data, and a computation unit configured to perform a computation based on the image data, and the imaging process is executed by the illumination unit and the imaging unit in a state in which the culture receptacle has been placed on the stage; and the computation unit executes the detection process, the calculation process, the determination process, and the area change calculation process based on the image data stored by the image data storage unit.

(8) Eighth Aspect

Furthermore, an eighth aspect of the present invention is any one of the first to the seventh aspects, wherein the epithelial cells are intestinal epithelial cells.

Namely, as long as the epithelial cells subject to the present invention have a fluid secretion function, there is no particular limitation thereto, such as corneal epithelial cells or respiratory tract epithelial cells. However, due to the method of establishing organoids shaped as crypts being established, the present invention is particularly appropriately applied to intestinal epithelial cells as the subject epithelial cells.

The above described aspects of the present invention enable simple evaluation of fluid secretion function of epithelial cells having a fluid secretion function such as intestinal epithelial cells in a short period of time without needing to perform staining using a fluorescent dye or the like.

### BRIEF DESCRIPTION OF DRAWINGS

FIG. 1A is a perspective view illustrating the external appearance of an image analysis device employed in an embodiment of the present invention.

FIG. 1B is a perspective view illustrating an image analysis device employed in an embodiment of the present invention when a hatch is in an open state (B).

FIG. 2 is an outline block diagram of an image analysis device employed in an embodiment of the present invention.

FIG. 3 schematically illustrates a measurement system of an image analysis device employed in an embodiment of the present invention.

FIG. 4 shows examples of images of objects being detected in an embodiment of the present invention.

FIG. 5 shows micrographs illustrating forskolin-induced swelling of a human intestinal organoid stimulated with forskolin.

FIG. 6 is a graph illustrating results for plural organoids over 240 minutes of observations of forskolin-induced swelling for human intestinal organoids stimulated with forskolin (10⁻⁵M).

FIG. 7 illustrates a comparison of images of forskolin-induced swelling for human intestinal organoids stimulated with forskolin (10⁻⁶M) prior to addition ((A) to (C)) and 30 minutes after addition ((D) to (F)).

FIG. 8 is a graph illustrating results observed for individual organoids of forskolin-induced swelling for human intestinal organoids stimulated with forskolin (10⁻⁶M).

FIG. 9 illustrates a swelling response induced by forskolin in a comparison of an average across three wells of rates of increase in total cross-sectional area of organoids for each well. The scale-bars at 10 minutes, 20 minutes, and 30 minutes indicate ±standard errors in the average values for the three wells.

FIG. 10 illustrates dose-dependency curves obtained for forskolin-induced swelling employing human small intestinal organoids. The dose-dependency curves exhibit a sigmoid shaped curve computed to have a log EC₅₀ value of −7.58. All results are an average of at least three independent tests.

FIG. 11 verified human intestinal organoid swelling induced by six different types of endogenous mediator related to anion/fluid secretion. The phase contrast images illustrated are prior to swelling induction and 60 minutes after swelling induction. Scale-bar: 100 μm. Induction was by the addition to a culture medium of: PGE₂ (10⁻⁸ M, (A)), VIP (10⁻⁷ M, (B)), ACh (10⁻³ M, (C)), histamine (10⁻³ M, (D)), bradykinin (10⁻⁵ M, (E)), and serotonin (10⁻³ M, (F)).

FIG. 12 illustrates dose-response curves for human intestine-derived organoids obtained by quantitative evaluation of swelling responses induced by PGE₂, VIP, ACh, and histamine using a 3D-scanning system.

FIG. 13 illustrates dose-response curves for human colonic organoids derived from a non-inflamed mucous membrane of an ulcerative colitis (UC) patent obtained by quantitative evaluation of swelling responses induced by PGE₂, VIP, ACh, and histamine using a 3D-scanning system. The organoids were established from plural samples of surgically obtained colon sections.

### DESCRIPTION OF EMBODIMENTS

Explanation follows regarding embodiments of the present invention, with reference to the drawings.

Image Analysis Device Outline

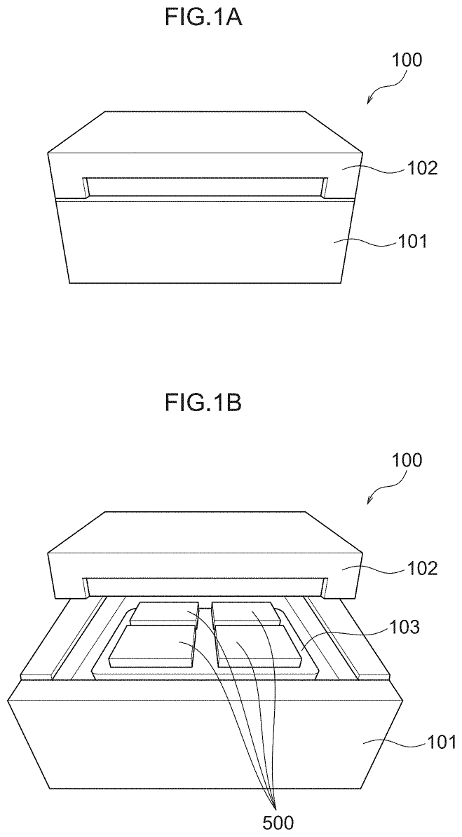

An image analysis device 100 according to a present embodiment has the external appearance as illustrated in FIG. 1A. Namely, a hatch 102 capable of opening and closing is provided to an upper portion of a casing 101. A stage 103 is visible when the hatch 102 is open, as illustrated in FIG. 1B. A maximum of four multi-well plates 500 serving as culture receptacles can be placed on the stage 103.

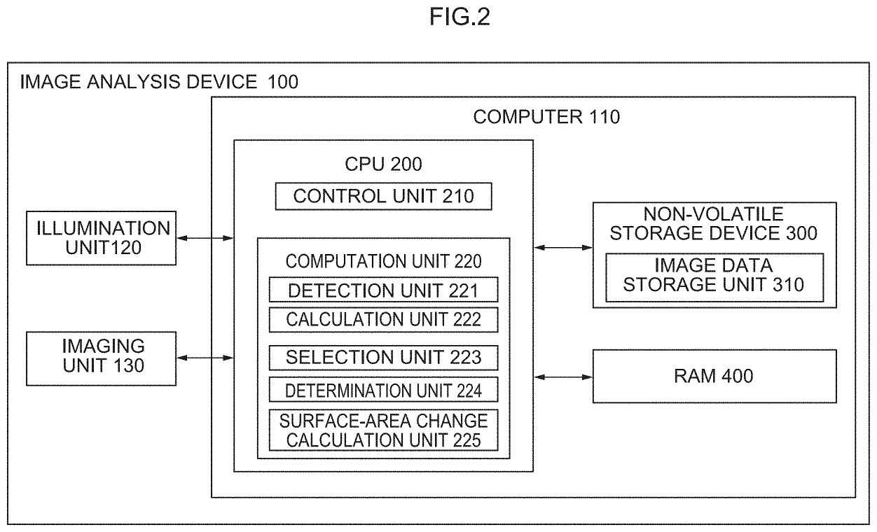

As illustrated in the block diagram of FIG. 2, the image analysis device 100 includes a computer 110, and an illumination unit 120 and an imaging unit 130 controlled by the computer 110. The computer 110 includes a CPU 200 for controlling the device overall, and, to store various data on, a non-volatile storage device 300, such as a hard disk or ROM, and RAM 400, serving as a volatile storage device.

The CPU 200 executes prescribed programs to function as a control unit 210 to control the image analysis device 100 in general (and in particular to control the illumination unit 120 and the imaging unit 130), and to function as a computation unit 220 to perform various computations based on image data obtained by the imaging unit 130.

The control unit 210 controls an imaging process performed by the imaging unit 130 while controlling the illumination unit 120 to provide appropriate illumination.

The computation unit 220 functions as a detection unit 221, a calculation unit 222, a selection unit 223, and a determination unit 224. The detection unit 221 executes a detection process to detect objects by determining edges of objects from image data. The calculation unit 222 executes a calculation process to calculate cross-sectional areas of objects. The selection unit 223 executes a selection process to select a detected object as being an organoid-derived object, or not. The determination unit 224 executes a determination process to determine whether or not a selected object is to be subjected to analysis. The computation unit 220 also functions as a surface-area change calculation unit 225 that executes a surface-area change calculation process to calculate a change with time in the cross-sectional area of the object as calculated by the calculation process.

The non-volatile storage device 300 includes an image data storage unit 310 that stores, as image data, multi-pixel images obtained by the imaging unit 130.

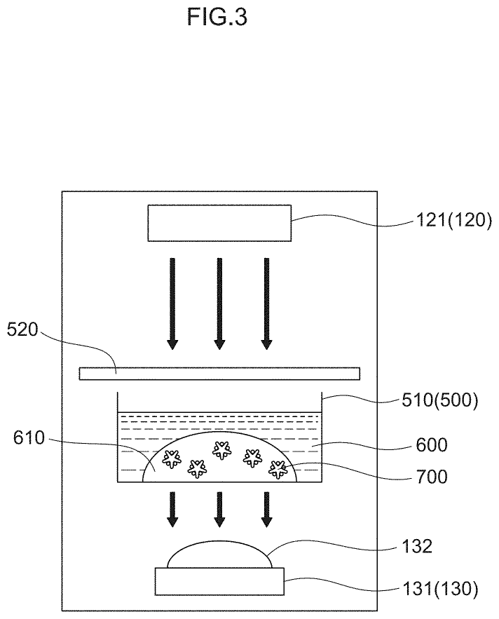

A measurement system of the image analysis device 100 is as schematically illustrated in FIG. 3. An LED white-light device 121 serving as the illumination unit 120 is installed above the multi-well plates 500, and a CCD cameral 131 serving as the imaging unit 130 is mounted below the multi-well plates 500. As described below, a culture gel 610 including organoids 700, and a culture medium 600 covering the culture gel 610 are employed to fill each well 510 of the multi-well plates 500. White-light emitted from the LED white-light device 121 that passes through lids 520 of the multi-well plates 500 also passes through the culture medium 600 and the culture gel 610 in the wells, and after being focused by a lens 132 below, is captured as a multi-pixel image by the CCD cameral 131. The organoids 700 in the culture gel 610 are recognized as objects by the difference in their refractive indices to that of the culture gel 610. Image data scanned at a resolution of 4,800 dpi can be obtained in this system within a minute for the whole of one of the multi-well plates 500.

Establishment and Culture of Human Intestinal Organoids

Human intestinal biopsy specimens were obtained from patients who underwent enteroscopic examination for the evaluation of diseases such as occult bleeding, irritable bowel syndrome, Crohn's disease and ulcerative colitis. Two or three biopsies were taken from an endoscopically normal region. Surgical specimens of ulcerative colitis patients were also collected to establish organoids. The present study was approved by the Ethics Committee of Tokyo Medical and Dental University and the Yokohama Municipal Citizens Hospital, and written informed consent was obtained from each patient. A total of 38 lines of small intestinal organoids (three lines of non-irritable bowel syndrome origin and 20 lines of irritable bowel syndrome (hereafter abbreviated to IBD) origin) or colonic organoids (four lines of non-IBD origin and eleven lines of IBD origin) were established from 27 patients. All the experiments were conducted in accordance with the approved guidelines. In the following, unless otherwise indicated, intestinal organoids established from the uninflamed mucosa of patients with Crohn's disease were mainly employed. Note that no organoids established from IBD patients with mucosa apparently diseased when viewed endoscopically were ever employed.

Isolation of crypts and the subsequent establishment of intestinal organoids were performed as previously described (Sato, T., et al., “Long-term expansion of epithelial organoids from human colon, adenoma, adenocarcinoma, and Bernett's epithelium.” Gastroenterology, 2011, 340: 1762 to 1772). Briefly, crypts were obtained by vigorously shaking biopsy specimens in 2.5 mM EDTA. Isolated crypts were embedded in 15 μl of Matrigel at a density of 20 to 30 crypts per well in either a 24-well or 48-well culture dish.

Those crypts were maintained in DMEM-based culture medium (Advanced-DMEM Invitrogen, California, USA) supplemented with recombinant human R-spondin-1 (1 μg/ml, R&D Systems, Minneapolis, USA), recombinant human Wnt-3 (300 ng/ml, R&D Systems, Minneapolis, USA), recombinant human Noggin (100 ng/ml, R&D Systems, Minneapolis, USA), recombinant human EGF (50 ng/ml, PeproTech, USA), Y-27632 (10 μM, Sigma-Aldrich Japan, Tokyo, Japan), A83-01 (500 nM, Sigma-Aldrich Japan, Tokyo, Japan) and SB202190 (10 μM, Sigma-Aldrich Japan, Tokyo, Japan). These culture conditions enabled the organoids to be maintained in an undifferentiated state, and thus allowed us to recognize the secretory function of human intestinal epithelial cells using those organoids.

Reagents Subjected to Analysis

The following were employed as reagents subjected to analysis: prostaglandin E₂ (PGE₂, Cayman Chemical, Michigan, USA), vasoactive intestinal polypeptide (VIP), acetylcholine (ACh), histamine, bradykinin and serotonin hydrochloride (Sigma-Aldrich Japan, Tokyo, Japan).

Organoid Cross-Sectional Area Measurement

The organoid cross-sectional area was measured using a 3D-Scanning System (Cell3 iMager, Screen Holdings, Kyoto, Japan) as the image analysis device 100. Organoids were seeded into a 96-well plate for 3D-scanning with 2 μl of Matrigel and 100 μl of complete culture medium. After the passage of a preliminary culture process of leaving for 24 hours after seeding, scanning was then performed both before and 30 min after the addition of the reagent subject to analysis. An image was acquired during scanning in auto-focus (AF) mode. The analysis parameters summarized in following Table 1 were set and the cross-sectional area of recognized organoids measured automatically.

TABLE 1

| | Setting | Default | |
|---|---|---|---|
| Parameter | Value | Value | Set range |
| Allowable object's | 25 | 40 | 0.1 to 100 |
| maximum area (%) | | | |
| Debris threshold (%) | 25 | 100 | 0 to 100 |
| Compactness upper | 200 | 300 | 0 to 500 |
| limit | | | |
| Edge detection | ON | OFF | ON or OFF |
| Edge-candidate | 20 | OFF | OFF or 0 to 120 |
| Edge threshold | 75 | OFF | OFF or 0 to 120 |
| Spheroid size lower | 1500 | 0 | — |
| limit (μm²) | | | |
| Spheroid size upper | 2400000 | 2147483647 | — |
| limit (μm²) | | | |
| Circularity lower | 45 | 0 | 0 to 100 |
| limit (%) | | | |
| Spheroid density | 30 | 300 | 0 to 400 |
| upper limit | | | |

A brief explanation will now be given regarding the parameters in Table 1.

The “allowable object's maximum area” is a threshold value for the size of an object up to which the object will be taken as being an organoid-derived object, expressed as a percentage of well area. An object lager than the setting value is not taken as being an organoid-derived object, and is not selected as such in the selection process.

The “debris threshold” is a threshold value to determine objects from “debris”. Namely, a portion having a difference in density to that of the background greater than the setting value is taken as being “debris”, and is not selected as an object in the selection process.

The “compactness upper limit” specifies an upper limit to a coherence (compactness C_{P}) of a region of an object. Namely, the longer a perimeter length P (a numerical value found by adding together not only the length of edges demarcating the inside and outside of an object, but also adding in the lengths of edges of voids when there are voids present in the object) with respect to the area (A) of the object, the greater the numerical value thereof, and the less cohesion there is in the region of the object. The value of the compactness C_{P} is 1 (100%) when an object is a perfect circle without any voids. When the setting value thereof is exceeded then this is taken as indicating “debris” and thus is not selected as an object in the selection process.

“Edge detection” is a parameter employed to facilitate object edge extraction by being switched ON when there are many objects present that are not readily detectable from the difference in density to the background density. The background density is an average pixel intensity value in a region sufficiently larger than the size of objects (for example, a 200 μm square region). The parameter settings for “Edge-candidate” and “Edge threshold” are enabled when “Edge detection” is switched ON.

Note that although not listed in Table 1, an “Include highlight area” parameter is also set to ON from a default value of OFF at the same time as “Edge detection” is switched ON. Setting the “Include highlight area” parameter to ON facilitates edge extraction even for regions having a particularly high brightness in the well images.

The “Edge-candidate” is a threshold value in an image for how small a difference in pixel intensity value can be to that of background for a pixel still to be taken as being an edge-candidate. This parameter is set to 20 in Table 1. Namely, in the present embodiment in which the pixel intensity values are expressed as 8 bit values, with a maximum value of 255 and a minimum value of 0, the edge-candidate parameter being set to 20 means a difference in pixel intensity value from the background is 7.8% (≈7.8125%=(20÷256×100). Hence pixels having a pixel intensity value from the background of the setting value or greater are taken as being edge-candidate pixels.

The “Edge threshold” is a threshold value for a sufficiently large difference in pixel intensity value from the background for a pixel to be detected as an edge in an image. This parameter is set to 75 in Table 1. In the present embodiment in which the pixel intensity values are expressed as 8 bit values, with a maximum value of 255 and a minimum value of 0, the edge threshold value parameter being set to 75 means a difference in pixel intensity value from the background is 29.2% (≈29.296875%=(75÷256×100). Hence pixels having a pixel intensity value from the background of the setting value or greater are recognized as confirmed-edge pixels.

A region surrounded by confirmed-edge pixels alone is detected as an object in the detection process. However, even when not surrounded by confirmed-edge pixels alone, if there are edge-candidate pixels present between the confirmed-edge pixels then a region surrounded by such pixels is also detected as being an object in the detection process.

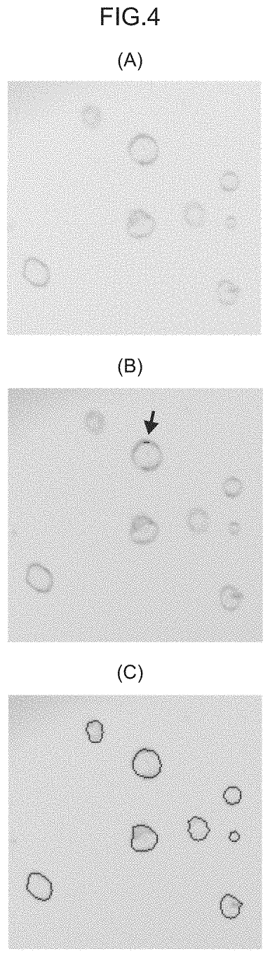

An example of object detection following parameter setting for “Edge detection” and “Include highlight area” is illustrated in FIG. 4. FIG. 4(A) is an image captured prior to performing edge detection. It is apparent that the pixel intensity values of outlines of objects in this image are not markedly different to the background. Only the location in this image indicated by the arrow in FIG. 4(B) is recognized as being an edge when both the “Edge detection” and “Include highlight area” are OFF. In contrast thereto, the outlines of all the objects are detected as clear edges when both the “Edge detection” and “Include highlight area” are ON, as illustrated in FIG. 4(C), resulting in a dramatic improvement in object detection capability. The areas of the regions surrounded by the edges detected in this manner are calculated as the areas of the respective objects.

The “Spheroid size lower limit” is a lower limit value of the area of a selected object for it to be taken as being a living organoid-derived object. Namely, an object for which the area of the region surrounded by the edge is less than the value of this parameter is taken as being derived from a dead organoid, and this object is removed in the determination process from being subject to analysis.

The “Spheroid size upper limit” is an upper limit value to an area of a selected object for it to be taken as being a living organoid-derived object. Namely, an object for which the area of the region surrounded by the edge exceeds the value of this parameter is also taken as being derived from a dead organoid, and this object is removed in the determination process from being subject to analysis.

The “Circularity lower limit” is a parameter indicating a lower limit value for the “circularity C_{R}” of an object. An object below this value has a distorted shape and so is taken as being derived from a dead organoid, and this object is removed in the determination process from being subject to analysis. A perfectly circular object has a circularity Ca value of 1 (100%).

The “Spheroid density upper limit” is a parameter to set an upper limit value of optical density (OD) for an object to be taken as being a living organoid-derived object. A lower limit value for the set range of zero represents a white pixel, and similarly an upper limit value of the set range of 400 represents a black pixel. Thus the setting value of 30 in Table 1 represents an upper limit value of optical density of 7.5% (=30÷400×100), and an object having an optical density exceeding this value is determined to be derived from a dead organoid, and is removed in the determination process from being subject to analysis.

By setting each of the parameters as described above, objects having clear edges are detected in the detection process, and the area of these respective objects is calculated in the calculation process. From out of these detected objects, those determined to be organoid-derived objects are selected by the selection process, and those objects that are further determined to be living organoids in the determination process are the objects finally subjected to analysis.

In the present embodiment, the organoids cultured in gel in the wells are suspended in the culture medium and do not move. Furthermore, there is no need to perform staining with a fluorescent dye cell staining product in order to perform observations. This thereby enables observations to be made of the changes over time in the cross-sectional area of the same organoids. Obviously statistical processing may be performed for plural organoids.

Note that changes over time due to the addition of a reagent subjected to analysis can be observed in the following manner. Namely, an object is imaged at time (T₀) prior to adding a reagent subjected to analysis (T₀ imaging process). Then the same object is imaged at time (T_{N}) when a prescribed time (30 minutes, for example) has elapsed since adding the reagent subjected to analysis. Determination is then made as to whether or not the object is an object subjected to analysis. By performing the selection process and the determination process, the change over time observations are only made for objects determined be subjected to analysis thereby at both time T₀ and time T_{N}.

Then in an area change calculation process, an area (A₀) at time T₀ and an area (A_{N}) at time T_{N} are calculated for the objects subject to analysis. The change over time (%) based thereon can be found, for example, using the following formula.

(A_{N}−A₀)/A₀×100

### EXAMPLES

The method for measuring the fluid secretion function of epithelial cells of the present embodiment is useful in practice for screening reagents.

Establishment of Quantitative Screening Method to Evaluate Swelling Response of Intestinal Organoids

First, forskolin-induced swelling was examined as a positive control in order to test the swelling response of human intestine-derived organoids. Intestinal organoids that were established from the uninflamed mucosa of patients with Crohn's disease were mainly employed in the present examples. These organoids were maintained under stem/progenitor cell-enriched culture conditions as an in vitro model of crypt cells.

The shape of the organoids was either multilobular or spheroidal depending on when the analysis was performed after the last passage. Organoids analysed within 2 days post-passage predominantly exhibited a spheroidal shape, while organoids analysed at 7 days or more post-passage exhibited a multilobular shape. These types of shape changes that occurred over time during the course of routine culture were repetitive and were not seen as variability among independent organoids at the same time point. Spheroidal organoids were employed in the present example using the 3D-scanner system.

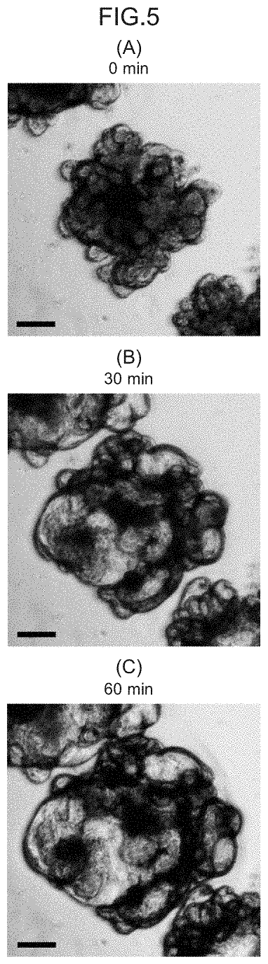

Consistent to former reports (Dekkers, J. F., “A functional CFTR assay using primary cystic fibrosis intestinal organoids.” Nature Med., 2013, 19(7): 939-945, and Foulke-Abel, J., et al., “Human Enteroids as a Model of Upper Small Intestinal Ion Transport Physiology and Pathophysiology” Gastroenterology, 2016, 150: 638-649), small intestinal organoids showed a rapid swelling response to the addition of forskolin, which continued for at least 60 min (see FIG. 5). Namely, swelling was observed that continued for 60 minutes in response to forskolin addition (10⁻⁵ M) using phase contrast images of intestinal organoids at 10 days after the passage.

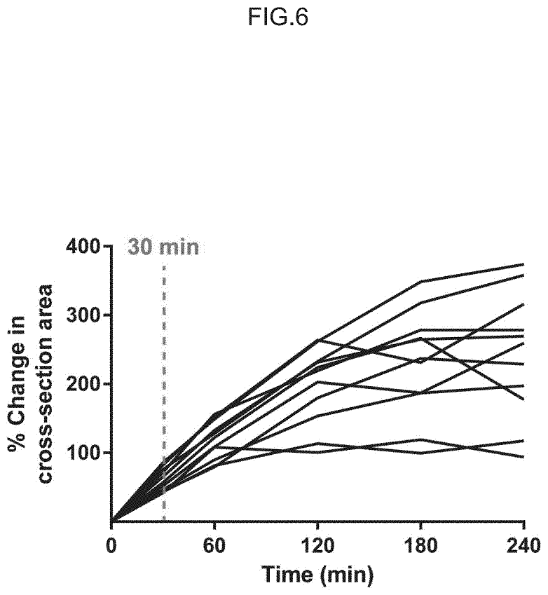

Further examination for up to 240 min revealed that organoids invariably show a continuous linear response up to 30 min after forskolin stimulation (see FIG. 6). Note that the numerical values in this graph are found by performing the area change calculation process of the embodiment. However, at longer than 30 min the response curve showed a heterogeneous pattern between the organoids. Some of the organoids exhibited a plateau pattern, suggesting that the response had reached equilibrium. Other organoids showed a declining curve, indicating a collapse of the organoids. It was determined from these preliminary data that measurements up to 30 min would be appropriate to examine the anion/fluid transport response of the organoids.

Using forskolin-induced swelling as a positive control, testing was next performed so see if such a swelling response could be quantified using the 3D-scanning system described. To optimize the quantification efficiency and accuracy, organoids were subjected to analysis after performing the preliminary culture process at one day after the last passage. This was done since the organoids generally held their cystic shape at this time point. The thresholds to recognize cross-section borders of each organoid were then optimized as set out in Table 1.

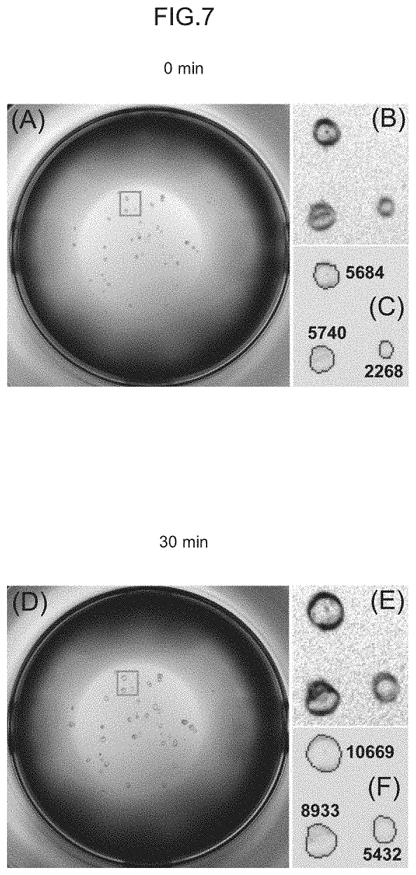

The optimized threshold settings were then confirmed to be compatible with accurate cross-section borders for each organoid (FIG. 7). FIG. 7(A) to (C) are images prior to forskolin addition. FIG. 7(D) to (F) are images at 30 minutes after addition. FIGS. 7(A) and (D) are images illustrating the whole of a well, and the rectangular regions therein are expanded in FIGS. 7(B) and (E), respectively. The edges detected by the detection unit in this state are respectively illustrated in FIGS. 7(C) and (F). These organoids are the same as each other, and the values of the areas of each of the organoids are indicated by the numbers appended adjacent thereto (units: μm²).

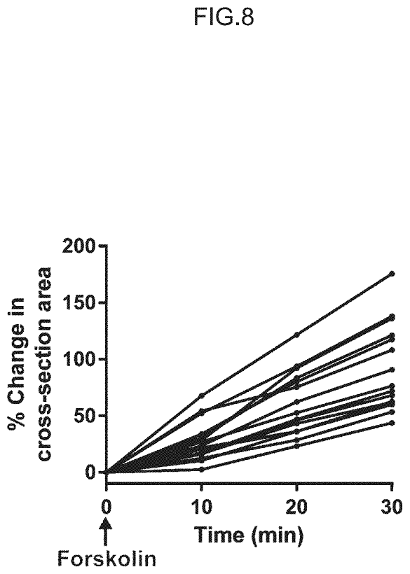

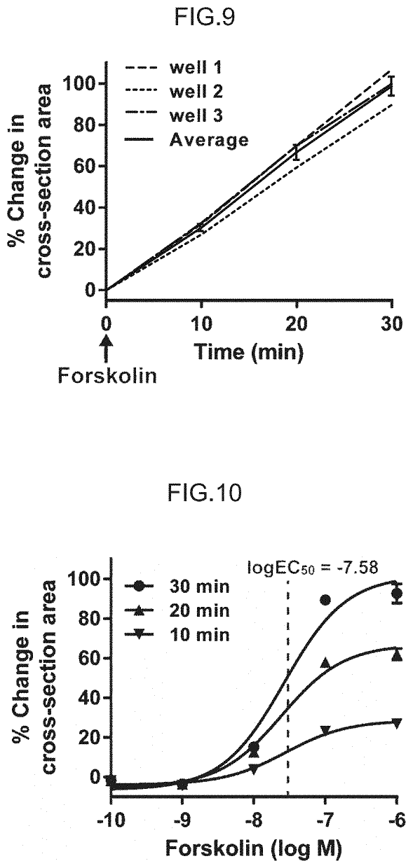

Namely, the cross-sectional area could be accurately measured by these settings. The increase in cross-sectional area of each organoid was calculated by scanning the organoids before and after forskolin addition, and these values are reported as an index of the swelling response. The time-dependent swelling response was confirmed, by using the present system, to be capable of being quantitatively monitored at the individual organoid level (FIG. 8). The response of individual wells was compared according to the method disclosed in Non-Patent Document 2 to evaluate the inter-well difference of forskolin-induced swelling (FIG. 9). It was apparent from the results that the proportional change in cross-sectional area derived from the total cross-sectional area of organoids in a single well is highly conserved among the individual wells tested under the same conditions.

The above confirmed that utilizing the fluid secretion function measurement method of the present embodiment enables the swelling response to be quantified for a maximum of 384 wells (96 wells/plate×4 plates) at a single scan.

Thus utilizing the fluid secretion function measurement method of the present embodiment enables drug response screening to be performed at the same time as determining the dose-dependency curves of a limited number of test reagents. Whether or not the present system could determine the dose-dependency curves of forskolin was thereby tested. The dose-dependency curve for forskolin-induced swelling when using a human jejunum-derived organoids was thereby found to be expressed by a standard sigmoid shaped curve, with the log EC₅₀ value thereof calculated as −7.58 (FIG. 10).

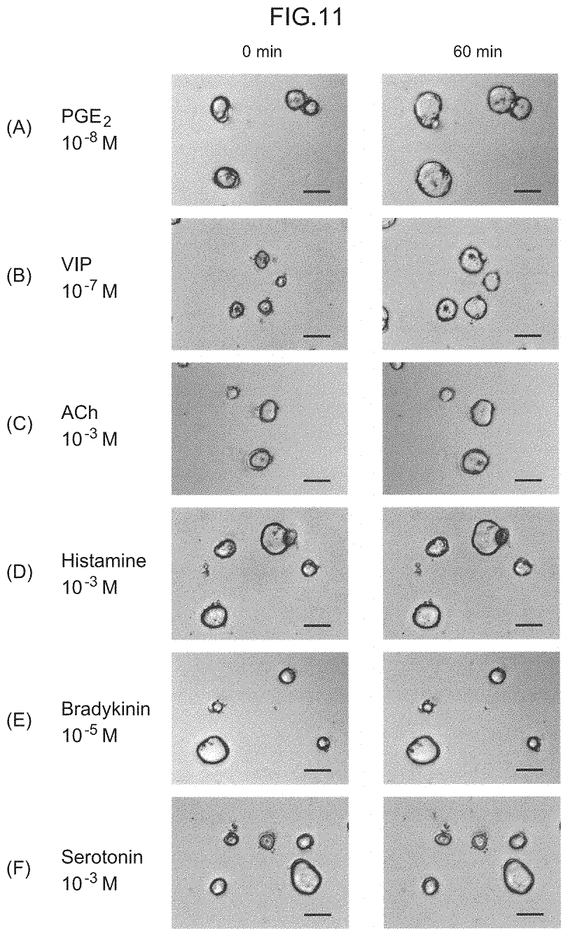

Examination of Endogenous Mediator Candidates for Anion/Fluid Secretion Next, the fluid secretion function measurement method of the present embodiment was employed to examine the direct effect of various endogenous mediators that might possibly induce anion/fluid secretion from intestinal epithelial cells by testing six types of endogenous mediators to determine if they had the capability to induce a swelling response in organoids equivalent to that of forskolin-induced swelling. Two types of mediator that function through Gs-coupled receptors, i.e. PGE₂ and VIP, exhibited a rapid and continuous response, ultimately resulting in a large overall increase in the cross-sectional area of the organoids at 60 min after induction (FIGS. 11(A) and (B)). In contrast thereto, mediators that function through Gq-coupled receptors, i.e. acetylcholine and histamine, showed a slow and limited response, and it was apparent that the increase in the cross-sectional area of the organoids was smaller than for PGE₂ or VIP (FIGS. 11(C) and (D)). The addition of bradykinin or serotonin did not exhibit a clear effect on the induction of organoid swelling (FIGS. 11(E) and (F)).

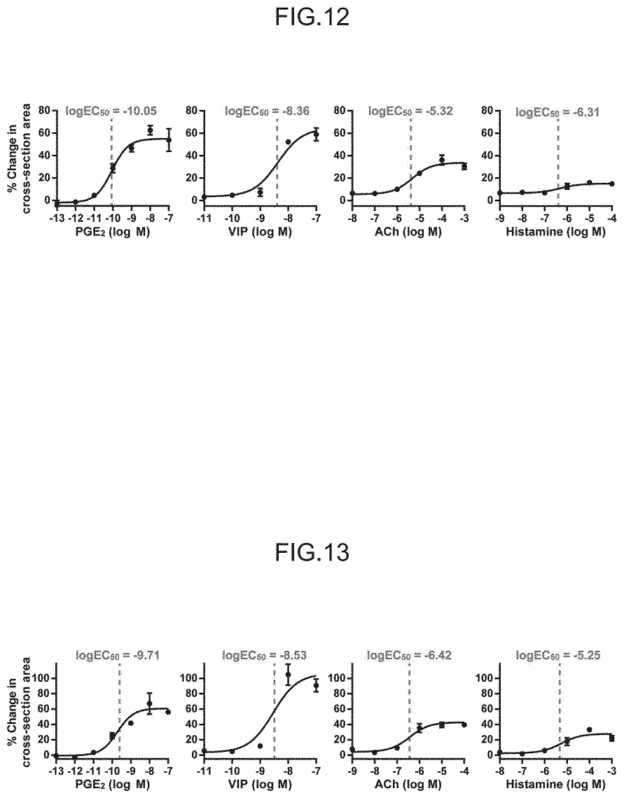

Next the differences in the mediator-induced dose-dependency curves of the organoids were examined to confirm the data acquired by time-lapse imaging. A human jejunal organoid was used first to evaluate the direct response of these mediators. The results were a clear sigmoid-shaped response curve exhibited for PGE₂ and VIP, while a markedly lower response profile was exhibited for acetylcholine and histamine (FIG. 12). This type of response pattern was also generally the same even in human colonic organoids derived from the uninflamed mucosa of a patient with ulcerative colitis (FIG. 13). Thus, these results showed that PGE₂ induced swelling of jejunal and colonic organoids at the lowest log EC₅₀ value of the mediators tested.

Therefore, among the various endogenous mediators of anion/fluid secretion, PGE₂ exhibited a strong function as a key inducer of anion/fluid secretion due to its direct effect on intestinal epithelial cells.

Thus as described above, it is apparent that the present embodiment enables precise and rapid screening to be performed for fluid secretion function of epithelial cells such as intestinal epithelial cells.

### INDUSTRIAL APPLICABILITY

The present invention may be employed as a method to measure the fluid secretion function of epithelial cells having a fluid secretion function, and may specifically be employed as a device or program to realize such a measurement method.

1. A method for measuring a fluid secretion function of epithelial cells having a fluid secretion function, the method comprising:
a preliminary culture process of seeding organoids established from subject epithelial cells in a culture receptacle and culturing the organoids in a gel contained in each well of the culture receptacle for a prescribed period of time;
an imaging process, performed after the preliminary culture process, of capturing a multi-pixel image of each organoid-containing well;
a detection process of detecting a region as an object in the multi-pixel image captured in the imaging process, the region being surrounded by edges configured by pixels having at least a prescribed difference in pixel intensity value with respect to that of a background, the background being an average pixel intensity value of a prescribed region having a size capable of containing an organoid subject to measurement;
a calculation process of calculating an area inside the edges for the object detected in the detection process;
a selection process of selecting whether or not the object detected in the detection process is derived from an organoid; and
a determination process of determining whether or not an object selected as being derived from the organoid in the selection process is subject to analysis,
wherein no cell staining product for staining the organoids is added when performing the imaging process.

2. The method for measuring a fluid secretion function of epithelial cells of claim 1, wherein, taking a difference between a pixel intensity value of maximum brightness and a pixel intensity value of minimum brightness as 100%, in the detection process:
a pixel having a difference in pixel intensity value from the background of 7.8% or greater is taken as an edge-candidate pixel;
a pixel having a difference in pixel intensity value from the background of 29.2% or greater is taken as a confirmed-edge pixel; and
a region surrounded by confirmed-edge pixels alone, or a region surrounded by confirmed-edge pixels and edge-candidate pixels, is detected as the object.

3. The method for measuring a fluid secretion function of epithelial cells of claim 1, wherein, in the selection process:
for a compactness C_{P} defined by C_{P}=P²/(4π·A), in which A (μm²) is an area of the object and P (μm) is a perimeter length of the object, an object having a compactness C_{P} of 2.0 or less is selected as being derived from the organoid.

4. The method for measuring a fluid secretion function of epithelial cells of claim 1, wherein in the determination process:
for a circularity C_{R} defined by C_{R}=(4π·A)/E², in which A (μm²) is an area of the object and E (μm) is an edge length of the object, an object having a circularity C_{R} of 0.45 or greater is determined to be subject to analysis.

5. The method for measuring a fluid secretion function of epithelial cells of claim 1, wherein:
the imaging process includes a T₀ imaging process executed immediately after the preliminary culture process, and a T_{N} imaging process executed subsequently to the T₀ imaging process after a prescribed period of time has elapsed from execution of a prescribed treatment in each of the wells; and
the method further comprises an area change calculation process to calculate, for an object determined by the determination process to be subject to analysis, a change in an area of the object according to the T_{N} imaging process with respect to an area of the object according to the T₀ imaging process.

6. The method for measuring a fluid secretion function of epithelial cells of claim 1, wherein:
the method employs an image analysis device that comprises:
a stage on which the culture receptacle is placed so as to be observable under conditions appropriate for imaging the organoid,
an illumination unit configured to illuminate each well from above the stage,
an imaging unit configured to capture rays of light emitted by the illumination unit that have passed through each well as a multi-pixel image,
an image data storage unit configured to store the multi-pixel image as image data, and
a computation unit configured to perform a computation based on the image data;
the imaging process is executed by the illumination unit and the imaging unit in a state in which the culture receptacle has been placed on the stage; and
the computation unit executes the detection process, the calculation process, and the determination process based on the image data stored by the image data storage unit.

7. The method for measuring a fluid secretion function of epithelial cells of claim 5, wherein:
the method employs an image analysis device that comprises:
a stage on which the culture receptacle is placed so as to be observable under conditions appropriate for imaging the organoid,
an illumination unit configured to illuminate each of the wells from above the stage,
an imaging unit configured to capture rays of light emitted by the illumination unit that have passed through each of the wells as a multi-pixel image,
an image data storage unit configured to store the multi-pixel image as image data;
a computation unit configured to perform a computation based on the image data, and
the imaging process is executed by the illumination unit and the imaging unit in a state in which the culture receptacle has been placed on the stage; and
the computation unit executes the detection process, the calculation process, the determination process, and the area change calculation process based on the image data stored by the image data storage unit.

8. The method for measuring a fluid secretion function of epithelial cells of claim 1, wherein the epithelial cells are intestinal epithelial cells.
